(19) United States
(12) Patent Application Publication
(10) Pub. No.: US 2023/0048160 A1
(43) Pub. Date: Feb. 16, 2023

(54) CONNECTED WATCH WITH ROTATING BEZEL

(71) Applicant: WITHINGS, ISSY LES MOULINEAUX (FR)

(72) Inventors: Manon NAVELLOU, PARIS (FR); Pierre-Arnaud DONNET, Hong Kong (CN); Samuel CLERC, Hong Kong (CN)

(21) Appl. No.: 17/869,581

(22) Filed: Jul. 20, 2022

(30) Foreign Application Priority Data
Aug. 10, 2021 (FR) 2108606

(51) Int. Cl. A61B 5/00; A61B 5/332; G04B 19/28; A61B 5/0245

(52) U.S. Cl. CPC A61B 5/681; A61B 5/332; G04B 19/283; A61B 5/0245

(57) ABSTRACT

A portable electronic device configured to be positioned on a users wrist, the portable device being configured to perform an electrocardiogram, ECG, the portable electronic device includes a watchcase, a case back, configured to be at least partially in contact with the skin of the wrist, a glass, a bezel, mounted on the watchcase and surrounding the glass, movable in rotation with respect to the watchcase, a first ECG electrode, made of conductive material, on the case back and configured to be in contact with the skin of the wrist, a second ECG electrode, made of conductive material, on the bezel, an ECG electronic module, electrically connected to the first ECG electrode and the second ECG electrode, and configured to receive and process electrical signals from a user and retrieved by the ECG electrodes, to perform an electrocardiogram.

### CROSS-REFERENCE TO RELATED APPLICATIONS

This application is a U.S. non-provisional application claiming the benefit of French Application No. 21 08606, filed on Aug. 10, 2021, which is incorporated herein by reference in its entirety.

### FIELD

The present invention relates to the field of connected watches and in particular to connected watches capable of performing an electrocardiogram (ECG or EKG). By watch, it is meant a wearable device, the preferred position of which is the wrist (“wrist wearable”). The present invention also relates to the field of so-called hybrid connected watches, i.e. connected watches having a visual appearance closer to conventional mechanical watches, in particular thanks to the presence of a gear train as well as mechanical hands to indicate at least the time (hour hand and minute hand).

### BACKGROUND

Watches that may perform an ECG (hereafter referred to as “ECG-watches”) are rare on the market in 2021. These include the Withings Move ECG, the Withings ScanWatch, the Apple Watch and the Samsung Galaxy.

An ECG is a test that studies how the heart works by measuring its electrical activity. An electrical impulse passes through the heart during each contraction and the ECG is a record of the electrical currents. The ECG is one of the main measurements for cardiovascular monitoring. Thanks to its integration in a watch, any user may regularly perform an ECG, which allows a better analysis and prevention of cardiac risks. To perform an ECG, several electrodes are placed on the human body to detect different components of the electrical signals, which are called leads. In one of its simplest forms, the ECG provides a bipolar measurement between the right and left arms, called lead I or DI.

The test is painless, passive and non-invasive (no injection of current into the skin) and may be performed in less than one minute. Although the concept of integrating an ECG into a consumer device has been presented for several years (see U.S. Pat. No. 5,289,824), actual implementation has only recently been successful. This is a major technological innovation.

ECG watches generally provide a DI ECG, although other leads may be obtained. For this purpose, an ECG watch comprises a watchcase, a case back, a glass, and either a dial (possibly with a screen or a digital display) or a display. A distinction may be made between smartwatch-type ECG watches and hybrid-type ECG watches. In the first case, the ECG-watch comprises a screen (for example an LCD screen) and the glass, instead of the traditional watch face and glass. In the second case, the ECG-watch comprises a dial, with hands and the glass (with or without a display integrated into the dial), thus giving a more traditional appearance to the whole. ECG watches may also include a bezel, which is a piece surrounding the screen or dial (and/or glass). Similarly, ECG-watches may include a crown, which radially passes through the watchcase (typically positioned between 1 and 5 o'clock) to enter a volume defined by it, and which allows interaction with electronic or mechanical components of the watch (menu activation, scrolling, etc.). The crown may be a pusher and/or a rotary wheel.

To perform an ECG with a watch, at least two electrodes (often three) are needed. They must be in contact with two different portions of the body. The document US2019246979A1 proposes to integrate the two electrodes to the strap, one on the inner side and one on the outer side. Other documents propose to mount a first electrode on the case back, in order to be in contact with the wrist on which the ECG-watch is mounted. The choice of the second electrode has been the subject of different technological orientations. US2020229761, on behalf of Apple, proposes to integrate the second electrode into the crown. Document PCT/EP2021/058955, in the name of Withings (and incorporated herein by reference) and not published on the day of filing of the present application, proposes to integrate the second electrode into the bezel. Other solutions exist, such as a second electrode positioned on the screen or next to the screen.

The implementation of an ECG measurement device in a watch presents many technical difficulties, particularly in that the electrical signals to be identified are of low amplitude and their acquisition is often noisy. The correct positioning of the electrodes and the management of the electrical chain between the electrodes and the electronic module that manages the ECG are crucial.

### SUMMARY

In a desire to diversify the range of products and to make connected watches as accessible as possible in terms of design and use, the inventors wanted to produce a model that respects as much as possible the aesthetic codes of DIVER-type watches, in particular (within the scope of the present description) of a connected watch with a rotating bezel. The benefits of a generalized use of an ECG-watch are multiple: the prevention of cardiac risks is improved, the financial cost on society is reduced and the state of scientific knowledge is multiplied by giving access to regular ECG data on thousands or millions of people (compared to punctual tests in hospitals or laboratories).

In addition, ECG watches usually have other functions (e.g. measurement of oxygen saturation SpO2 or heart rate by PPG), so that these watches have a higher research value by allowing the data to be combined.

An aspect of the present invention relates to a portable electronic device, configured to be positioned on a user's wrist, the portable device making it possible to perform an electrocardiogram, ECG, the device comprising:
a watchcase,
a case back, configured to be at least partially in contact with the skin of the wrist,
a glass,
a bezel, mounted on the watchcase and surrounding the glass, movable in rotation with respect to the watchcase,
a first ECG electrode, made of conducting material, on the case back and configured to be in contact with the skin of the wrist
a second ECG electrode, made of conductive material, on the bezel,
an ECG electronic module, electrically connected to the first ECG electrode and the second ECG electrode, and configured to receive and process electrical signals coming from a user and recovered by the ECG electrodes, in order to perform an electrocardiogram.

The portable electronic device may be an electronic watch.

In an embodiment, the bezel comprises a bezel body and the second ECG electrode is formed by the bezel body, such that the entire bezel body forms the second electrode and the user may touch any portion of the bezel body to perform an ECG measurement.

In an embodiment, the second ECG electrode is electrically connected to the ECG module regardless of the angular position of the bezel.

In an embodiment, the device comprises an electrical connector in the bezel configured to electrically connect the bezel and the ECG module, the electrical connector providing the connection between the ECG module and the electrode.

In an embodiment, the electrical connector is removably mounted on the watchcase.

In an embodiment, the electrical connector comprises a compression spring configured to make electrical contact with the bezel.

In an embodiment, the compression spring is a leaf spring and/or the electrical connector comprises a plurality of angularly spaced compression leaf springs.

In an embodiment, the electrical connector comprises a ring positioned around the glass and/or a dial of the device.

In an embodiment, the connector comprises feet, and wherein the leaf springs extend from one side of the ring and the feet extend from another side of the ring.

In an embodiment, the feet comprise lugs and/or tabs.

In an embodiment, the feet are electrically connected with the ECG electronic module, so that the ECG signal passes through at least one of the feet.

In an embodiment, the compression spring exerts pressure on the foot so as to maintain the electrical connection of the foot to the ECG electronic module.

In an embodiment, the electrical connector comprises a conductive coating, such as gold.

In an embodiment, the bezel comprises, on an inner face, a series of detents adapted to interact with the spring in compression, the series of detents and the spring allowing to define a stepwise displacement for the rotation of the bezel.

In an embodiment, the watchcase comprises a bezel holder, the bezel being rotatably mounted on the bezel holder and the electrical connector being mounted on the bezel holder.

In an embodiment, the bezel holder is electrically connected with the ECG electronic module, so that the ECG signal passes through the bezel holder.

In an embodiment, the glass is mounted on the watchcase or on the bezel.

In an embodiment, the watchcase comprises a main body, the bezel holder, and an annular seal between the bezel holder and the main body of the watchcase, the seal enabling the bezel holder to be held in position and/or the bezel holder to be electrically isolated from the main body of the watchcase.

In an embodiment, the bezel comprises serrations on an outer side face, the serrations forming part of the second electrode.

In an embodiment, the device further comprising a wireless communication module, configured to bidirectionally communicate with a mobile terminal, for example to send ECG results obtained by the ECG module.

In an embodiment, the portable electronic device is a watch, the watch being for example a hybrid watch with mechanical hands.

In an embodiment, the case back comprises an optical sensor configured to emit and receive light.

In an embodiment, the bezel is a substantially rotationally symmetrical part.

In an embodiment, the bezel is movable in counterclockwise rotation only, in particular thanks to the electrical connector and its compression springs.

In an embodiment, the spring works in a direction parallel to the axis of rotation of the bezel; in an embodiment, the spring works in a direction orthogonal to the axis of rotation of the bezel.

An aspect of the present invention also relates to a method of taking an electrocardiogram, ECG, measurement using a device as described above, during which the user brings one arm into contact with the first electrode and another arm into contact with the second electrode. By “arm” is meant the upper limb extending from the shoulder to the hand. In particular, the device is an ECG watch, worn on a wrist so that the first electrode is in contact with the wrist and the user touches the rotating bezel with the other hand.

Another aspect of the present invention also relates to a use of a device as previously described for performing an electrocardiogram.

### BRIEF DESCRIPTION OF THE DRAWINGS

Further features, details and benefits will become apparent from the following detailed description, and from an analysis of the attached drawings, in which:

FIG. 1.

This figure shows a top view (along a Z direction) of the ECG-watch, where the dial, the glass and the bezel in particular are visible, according to an embodiment (the hands are not shown).

FIG. 2

This figure shows a view from below (along a Z direction) of the ECG-watch, where the case back and the watchcase in particular are visible, according to an embodiment.

FIG. 3

This figure shows a side view (along an X direction) of the ECG-watch, according to an embodiment of the hybrid ECG-watch.

FIG. 4

This figure shows a top view (along a Z direction) of the ECG watch, where the glass is visible, according to a non-hybrid watch embodiment.

FIG. 5

This figure shows a three-dimensional (partially transparent) view of the ECG-watch, according to an embodiment (lens or protective glass of the optical sensor not shown).

FIG. 6

This figure shows a cross-sectional view along the XZ plane of an ECG-watch, according to an embodiment.

FIG. 7

This figure shows a three-dimensional view and an enlargement of an electrical connector, according to an embodiment.

FIG. 8

This figure shows a three-dimensional view and enlargement of an electrical connector, according to another embodiment.

FIG. 9

This figure shows a three-dimensional view of the bezel, with the bottom side visible.

FIG. 10

This figure shows a cross-sectional view, in which the engagement of the electrical connector with the notches of the bezel is particularly visible.

FIG. 11

This figure shows a three-dimensional view, in which the engagement of the electrical connector with the notches of the bezel is particularly visible.

FIG. 12

This figure shows a three-dimensional view of a bezel holder.

FIG. 13

This figure shows a cross-sectional view of an ECG-watch, according to an embodiment in which a foot of the electrical connector is in the form of a lug.

FIG. 14

This figure shows a cross-sectional view of an ECG-watch, according to an embodiment where the electrical connector comprises a spring and a ball bearing.

FIG. 15

This figure shows a three-dimensional view of a holding ring between the bezel and the bezel holder (not shown).

FIG. 16

This figure shows a cross-sectional view of an ECG-watch, according to an embodiment, where certain components of the electrical chain for the ECG are particularly visible.

FIG. 17

This figure shows another cross-sectional view of an ECG-watch, at 90° to that of FIG. 16, according to the same embodiment, where some components of the electrical chain for the ECG are particularly visible.

FIG. 18

This figure shows a diagram of the ECG-watch with some components, especially electronic components.

FIG. 19

This figure shows comparative test results with the Withings ScanWatch 38 mm, which has a fixed ECG electrode bezel.

FIG. 20

This figure shows a three-dimensional view of a bezel holder comprising an alternative electrical connector and rotative bezel embodiment.

FIG. 21

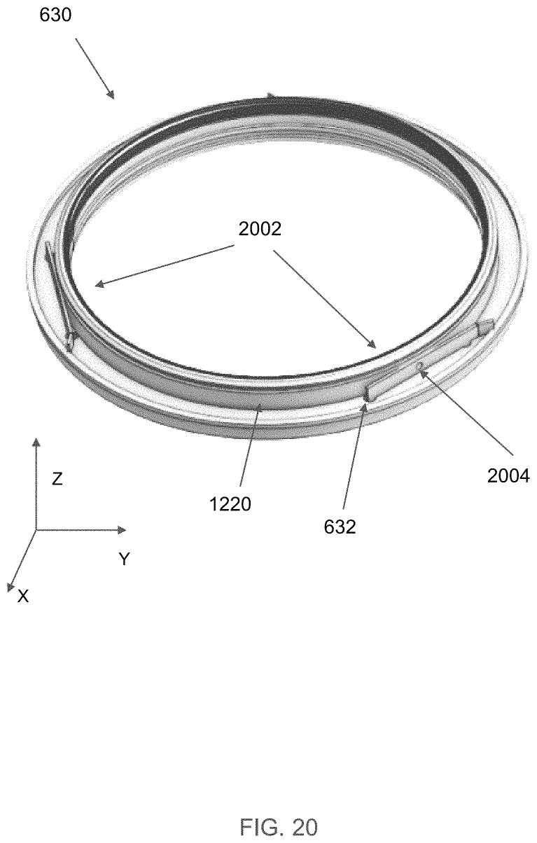

This figure shows a detailed view of the electrical connector of FIG. 20 cooperating with notches of the bezel.

### DETAILED DESCRIPTION

Examples of embodiments of an ECG-watch according to the present application are described in this section. The examples are provided to illustrate and better understand the various embodiments.

An aspect of the present description relates to a portable electronic device comprising an electrocardiogram sensor (hereinafter: ECG sensor). In a particular embodiment, which is the one illustrated, the portable electronic device is a watch (hereinafter: ECG-watch). The ECG-watch may comprise a wristband. Nevertheless, in the context of the present description, the term ECG-watch does not necessarily include the strap, which is generally manufactured elsewhere and may be assembled at points of sale.

The portable electronic device is connected, so that it may exchange data remotely (wirelessly) in a bidirectional way with a terminal, such as a smartphone. The connection may be made via Bluetooth® (which is a wireless technology that enables the exchange of data between devices using wavelength), such as Bluetooth® Low Energy (BLE), (which is designed for very low power operation. BLE transmits data over 40 channels the 2.4 GHz unlicensed industrial, scientific and medical (ISM) frequency band). In particular, the data exchanged from the ECG-watch to the terminal is ECG data acquired by the ECG-watch. The ECG-watch may also receive data from the terminal (time, alarm data, notifications, etc.).

In an embodiment, the ECG-watch is a hybrid watch, i.e. a watch with a dial and hands to indicate the hours and minutes.

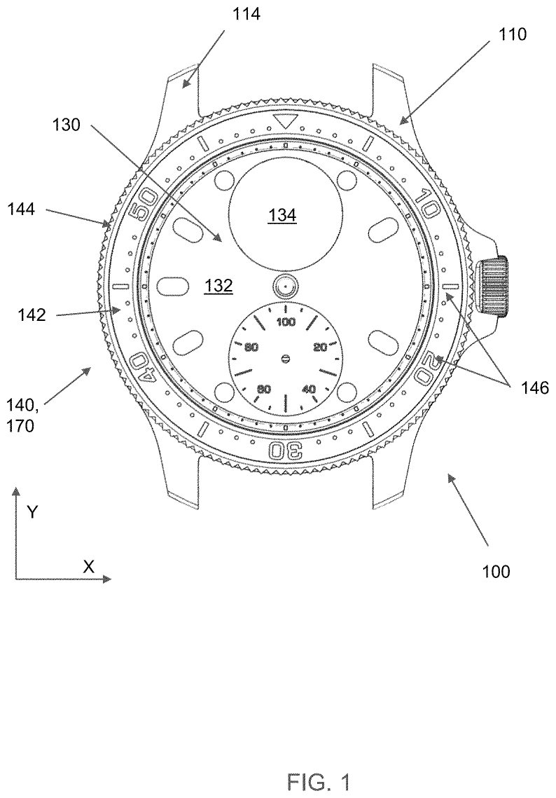

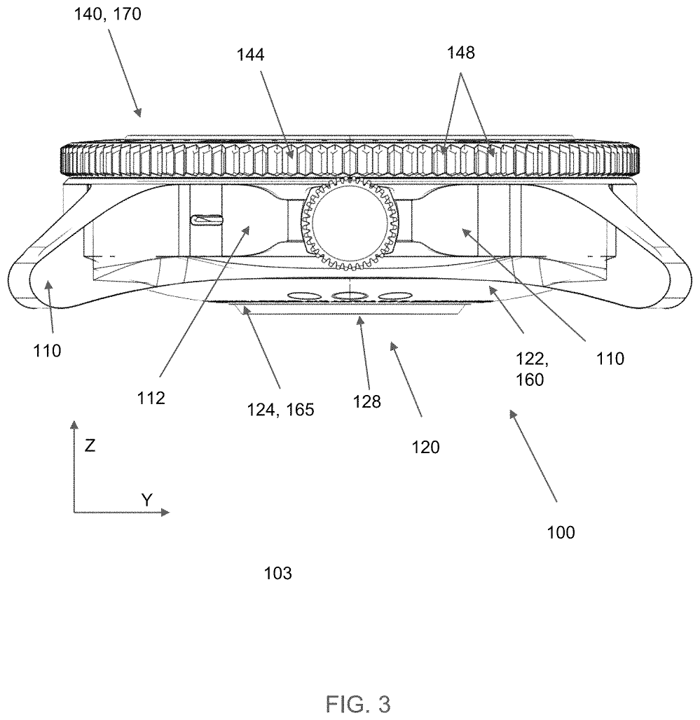

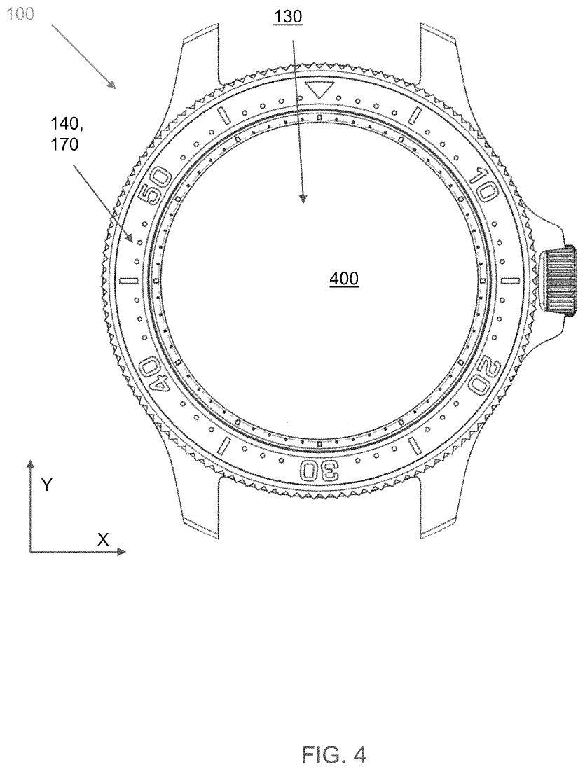

FIGS. 1 to 3 illustrate an electronic ECG-watch 100, of the hybrid type (with a dial, mechanical hands, and possibly a display integrated into the dial). FIG. 4 illustrates a non-hybrid ECG-watch 100 (without mechanical hands but with a display). A standard reference mark (XYZ) is shown in these figures. In the present description, the notion of “top” and “bottom” is defined in relation to the Z direction, the top being in the direction of the glass and the bottom being in the direction of the case back, which will be described below.

The ECG-watch 100 may include a watchcase 110 and a case back 120 that is configured to at least partially contact the skin of the users wrist. The watchcase 110 and the case back 120 are integral with each other. In an embodiment, as illustrated in the figures, the watchcase 110 and the case back 120 are two separate pieces. In an embodiment not shown in the figures, the case back 120 is a part integrated with the watchcase 110. The watchcase 110 may include a side wall 112, which is generally visible when the ECG-watch 100 is worn on the wrist. The watchcase 110 may include lugs 114 (two pairs, on either side of the watchcase 110) for attaching a strap (not shown in the figures). The watchcase 110 may include a plurality of parts.

The ECG-watch 100 may also include a glass 130, typically mounted on the watchcase 110, such that the glass 130 is fixed. The glass 130 is or may comprise a typically transparent protective glass and may be made of organic or inorganic glass, ceramic, plastic or any transparent material. The outline of the glass 130 is here typically circular in shape.

In the case of a hybrid watch, below the glass 130, the ECG-watch 100 further comprises a dial 132 with hands (physical hands, which are not shown in the figures). The dial 132 may further accommodate a display 134 (e.g., with an opening in the dial that allows a display positioned just below the dial to be visible), which occupies, for example, a small space below or within the dial 132. The glass 130 protects these parts and allows them to be seen through.

In the case of an Apple Watch type “smartwatch”, under the glass 130, the ECG-watch 100 comprises a screen 400 that occupies a width close to the width of the ECG-watch 100. In an embodiment, the screen 400 may display hands. The glass 130 is then the protective glass of the display 400.

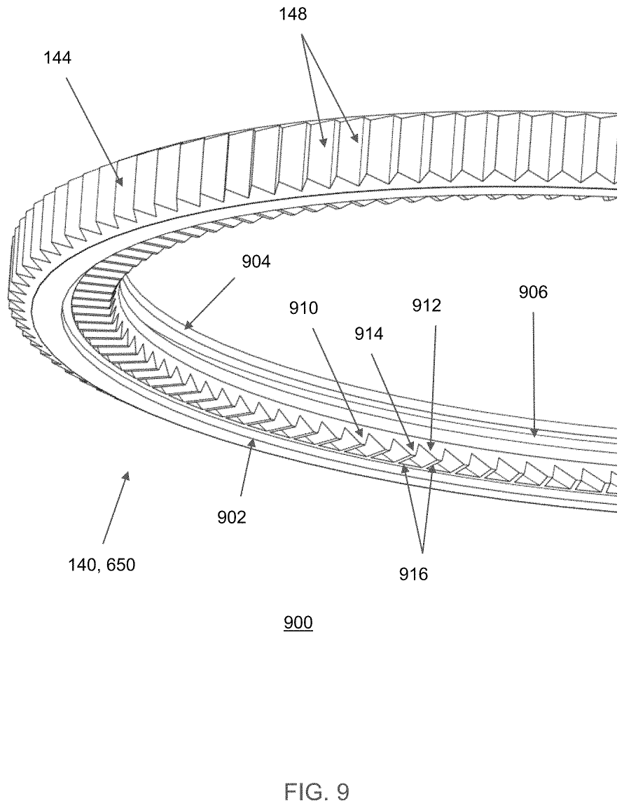

The ECG-watch 100 further comprises a bezel 140, mounted on the watchcase 110. The bezel 140 is positioned around the glass 130 (radially external to the glass). The bezel 140 is rotatably mounted with respect to the watchcase 110. The rotation may be step by step (for example, notch by notch). The number of steps may be sixty or one hundred and twenty steps (which thus correspond angularly to one or two notches per minute, an hour having 60 minutes which represent 360°). Any step value may be used (e.g. multiples of 15, 30 or 60). The user may thus rotate the bezel 140, in a manner classically known on a “DIVER” type watch. The bezel 140 may include an upper face 142, a lower face (not visible in FIGS. 1 to 3 but to be described in connection with FIG. 9), an outer side face 144 and an inner side face (not visible in FIGS. 1 to 3 but to be described in connection with FIG. 9). The upper side 142 and the outer side 144 are visible under normal conditions of use, whether or not the ECG-watch 100 is worn on the wrist.

The top surface 142 may include visual markers 146, such as graduations, numbers, etc. The purpose of these visual markers 146 is to assist the user in managing time. For example, if at a time t, a person wishes to know a time that is about to elapse, it is sufficient to align a visual marker 146 (for example an arrow or a dot that indicates 0) with the minutes. Thereafter, one need only look at the visual marker 146 to which the minute hand is pointing to know the elapsed time from time t. The outer side face 144 may include serrations 148 to facilitate the users hand gripping the bezel and thus rotating the bezel 140. In traditional “DIVER” watches, the bezel 140 allows the user to quickly estimate the elapsed dive time.

The bezel 140 may be rotated clockwise, counterclockwise or both. In the case of a traditional “DIVER” type watch, only counterclockwise rotation is permitted, so that a false manipulation will only increase the elapsed diving time and not decrease it.

The bezel 140 is a ring-shaped part, essentially of revolution about the Z direction (with some modifications). More details will be given below.

In a variant not shown and less common in DIVER watches, the glass is mounted on the rotating bezel, so that the glass moves with the bezel.

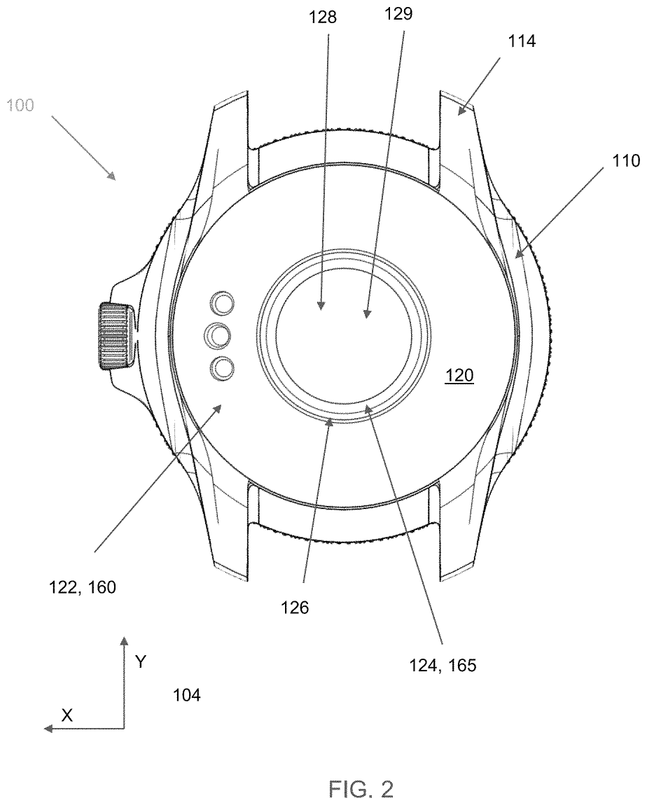

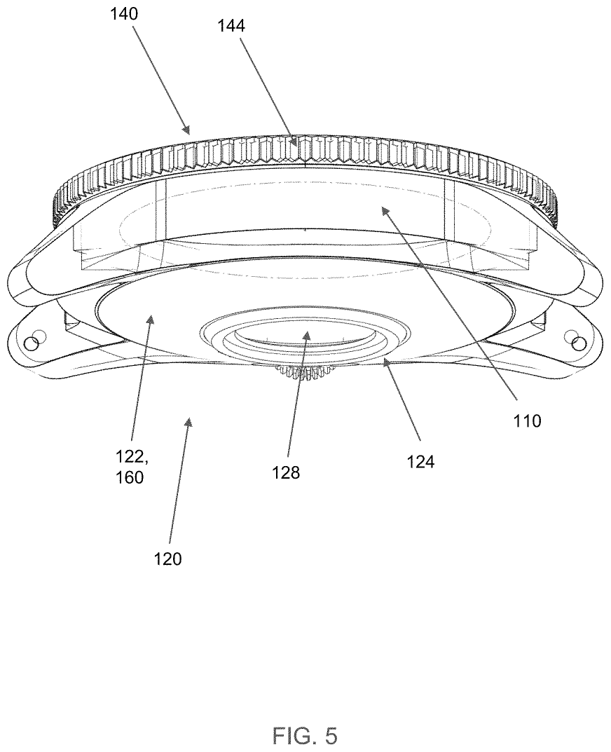

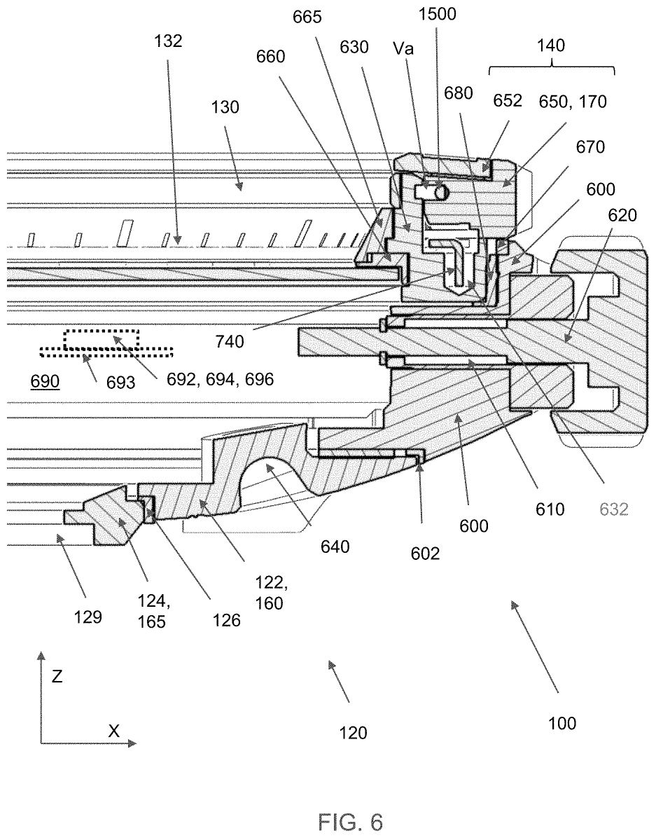

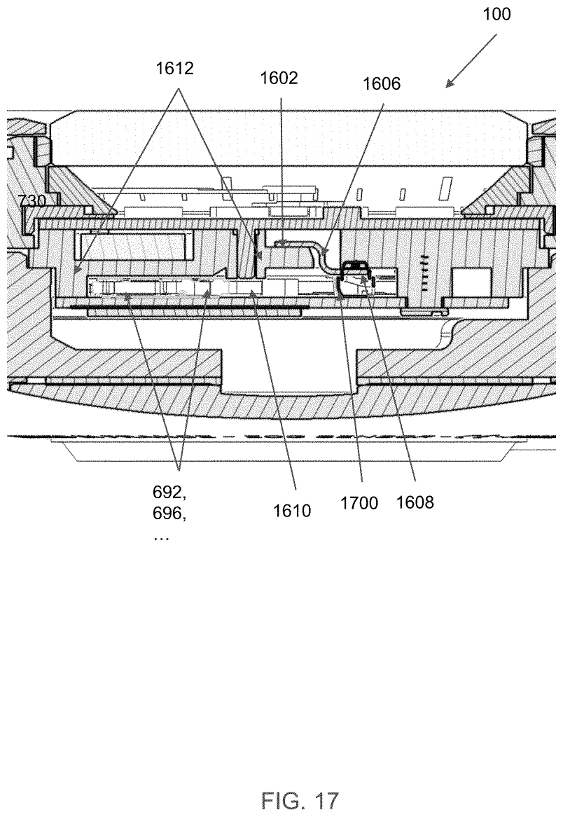

To retrieve electrical signals generated by the human body, the ECG-watch 100 comprises an ECG sensor. In particular, the ECG sensor comprises a set of electrodes (referred to as ECG electrodes) and an ECG electronic module 692 (illustrated very schematically in FIG. 6 but better visible in FIG. 17), to which the ECG electrodes are electrically connected. By electrode is meant a conductive part capable of receiving an electric current or voltage. The part may be made of a conductive material or comprise a conductive coating. By “conductor” is meant “electrically conductive”. As seen in FIGS. 2, 3, 5, 6 a first electrode 160 is located on the case back 120 so as to be in contact with the skin of the users wrist on which the user is wearing the ECG-watch 100. The first electrode 160 is electrically connected to the ECG module 692.

For example, the case back 120 may include an outer annular member 122 and an inner annular member 124 radially inward of the outer annular member 122. The outer annular member 122 is the first electrode 160. The two outer annular members 122 and 124 may be separated by a seal 126. The inner annular member 124 may surround an optical sensor 128, with at least one LED, for emitting light, and at least one photodiode, for receiving light. The inner annular member 124 may be protruding (and have a frustro-conical shape). The optical sensor 128 is typically a PPG (photoplethysmography) sensor. The optical sensor 128 may be positioned behind a lens 129, such as a glass lens, which interfaces with the skin of the wrist. PCT/EP2021/058955, in the name of Withings, and incorporated by reference, describes in detail the optical sensor, which is found on the Withings ScanWatch.

As seen in FIGS. 1, 3, 4-6, a second ECG electrode 170 is located on the bezel 140 so that it may easily contact the skin of the users other hand. The second electrode 170 is also electrically connected to the ECG module 692.

In an embodiment, the second electrode 170 is the bezel 140, i.e., the entire bezel 140 or a portion of the bezel (accessible by the user) forms the electrode 170. The user may thus touch the bezel at any point. Compared to a crown acting as an electrode, the ECG-watch 100 may be worn on the left and right hand equally, without the gestures to be performed to perform an ECG being the same for a user wearing the watch on the left or right hand (symmetry of use), unlike the electrode crowns described in the introduction. The integration of an electrode on a rotating bezel is counter-intuitive due to the rotation of the bezel and to the extent that other parts of the watch seem more appropriate, such as the crown. Nevertheless, the inventors found that it was possible to obtain good quality signals despite the difficulties related to the electrical chain, which are generated by the relative mobility between the bezel and the watchcase.

In particular, the second electrode 170 is electrically connected regardless of the angular position of the rotating bezel 140. Thus, the user does not need to put the rotating bezel 140 in a particular position to perform an ECG. As previously mentioned, the rotation may be by notch, into which the rotating bezel 140 moves. For each notch, the electrical connection may be made. More specifically, including between each notch, the electrical connection may also be made. Thus, there is full continuity of electrical connection between the second electrode 170 and the ECG module 692. In particular, the action to be performed to take an ECG is the same regardless of the position of the rotating bezel 140. In other words, the second electrode 170 is functionally invariant by rotation of the rotating bezel 140.

The electrodes 160, 170 are made of conductive material. In the embodiment shown in FIGS. 1, 3, 4 to 6, the electrodes are formed by the parts themselves, which are made of conductive material. Alternatively, the electrodes, in particular the first electrode, may be formed by a conductive coating deposited on a surface (which is itself conductive or non-conductive).

FIG. 6 illustrates a cross-sectional view of the ECG-watch 100 according to an embodiment. In particular, the watchcase 110 comprises a main body 600 (or watchcase body), which comprises the side wall 112. In FIG. 6, the main body 600 comprises an opening 610 at the level of the side wall 112 to allow the placement of a crown 620. Here, the crown 620 has a role only as a user interface between the ECG-watch 100 and the user. To this end, the crown 620 may be rotatable (here about the X axis) and/or translatable (here along the X axis). The crown 620 does not play a role for the ECG and will not be described further. The watchcase 110 may be mounted on the main body 600. As described above, the watchcase 110 may include the inner ring or inner annular member 124, the outer ring or outer annular member 122, the seal 126, and the lens 129. A gasket 602 may be provided between the watchcase 120 and the watchcase 110 (more specifically, between the outer annular member 122 and the main body 600 of the watchcase 110). In the cross-sectional view of FIG. 6, the outer annular member 122 comprises a recess 640, which is a positioning pad for a battery charging station. The watchcase 110 may also include a bezel holder 630, mounted to the main body 600. In particular, the bezel 140 is mounted on the bezel holder 630 in a radially outer position. The glass 130 is also mounted on the bezel holder 630, in a radially inner position.

The bezel 140 may include a bezel body 650, which is mounted to the watchcase 110. The bezel body 650 may directly incorporate (by engraving or otherwise) the traditional decoration of a watch, such that the bezel 140 is integrally formed by the bezel body 650. Alternatively, as illustrated in FIG. 6, the bezel 140 further comprises a display ring 652 (also referred to as a “decoration”) which may be mounted (e.g., by bonding, such as with double-sided adhesive) to a top side of the bezel body 650. The decor then comprises the aforementioned visual markers 146. The bezel body 650 is the second electrode 170, such that the user may touch any portion of the bezel body 650 (including the serrations 148) to perform an ECG. Any portion of the bezel body 650 means any portion of the surface of the bezel body 650 accessible to a user by touching.

The bezel body 650 may be made of a conductive material, such as metal (e.g., stainless steel or titanium alloy). Alternatively, the bezel body 650 may be made of filled plastic or conductive ceramics. Alternatively, the bezel body 650 may be non-conductive and a conductive coating is deposited on all or a portion of the bezel body 650 (e.g., from the top side or the outer side to the notches on the bottom side).

Various gaskets 660, 670, 680 are arranged between the various components to provide sealing, part retention and/or electrical insulation. The seals 660, 670, 680 have annular or circular shapes. They are of revolution about the Z direction. A dial gasket 660 provides electrical insulation between the bezel holder 630, a flange 665 and the dial 132. A protective gasket 670 disposed between the main body 600 of the watchcase 110 and the bezel 140 limits the introduction of dust into the ECG-watch 100. In addition, the protective gasket 670, by preventing debris from becoming trapped and creating an electrical connection, assists in providing effective electrical insulation between the bezel 140 and the watchcase main body 600. In the event that debris becomes trapped between the bezel 140 and the watchcase 110 of the body 600. The protective seal 670 also serves as a mechanical stop for the bezel 140 along the Z-direction. A seal 680, disposed between the bezel holder 630 and the main body 600 of the watchcase 110, electrically insulates the bezel holder 630 from the main body 600 and maintains the parts in position (shock resistance in particular). In addition, the seal 680 provides a seal at 10 atmospheres (atm), which the protective seal 670 does not generally provide (as it is not a compression seal). The seal 680 typically has an L-shaped cross-section (visible in FIG. 6). In the figures, the radially outer walls of the gasket 680 contact a side extension and flat of the main body of the watchcase and the radially inner portions of the gasket 680 contact a corner of the bezel holder 630.

The watchcase 110 (and in particular the case back 120, the dial 132 and the main body 600 of the watchcase 110) defines an internal volume 690 suitable for receiving various components, such as electronic components. These electronic components are thus protected from water or dust (with the appropriate seals, in particular permitted by the aforementioned seals).

In order to enable an ECG to be taken, the ECG-watch 100 comprises an ECG module 692 which is housed in the internal volume 690 (schematically shown as dotted line in FIG. 6 and schematically positioned). The two electrodes 160, 170 are electrically connected to the ECG module 692. The ECG module 692 is configured to retrieve electrical signals from the human body and to, after processing, generate an electrocardiogram. The ECG module 692 may be mounted on an electronic board 693 (schematically shown as a dotted line in FIG. 6 and schematically positioned), such as a printed circuit board (PCB), where other components of the ECG watch are also mounted.

The optical sensor 128 is connected to a PPG module 694, also positioned in the internal volume, which may also be mounted on the electronic board 693 of the ECG-watch 100. The PPG module 694 is configured to generate the instructions for the LEDs and to recover the electrical signals from the photodiodes.

The control unit 696 is used to control the on-board electronics of the ECG-watch 100. The control unit 696 may for example include or partially include the ECG module and the PPG module.

The ECG electrode assembly may include a third electrode 165, for example, on the case back 120. In particular, the inner ring or inner annular member 124 may be the third electrode 165. PCT/EP2021/058955, in the name of Withings, describes the arrangement of the first and third electrodes. The third electrode may serve as a reference to the other two electrodes. Alternatively, the first electrode 160 may be the inner ring or inner annular member 124 and the third electrode, if applicable, the third electrode may be the outer ring or outer annular member 122.

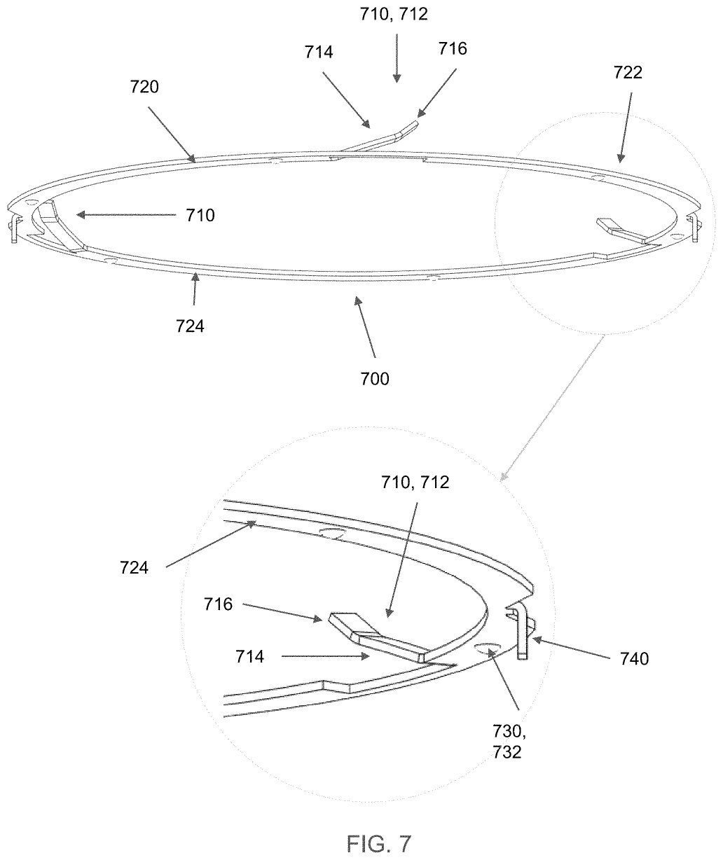

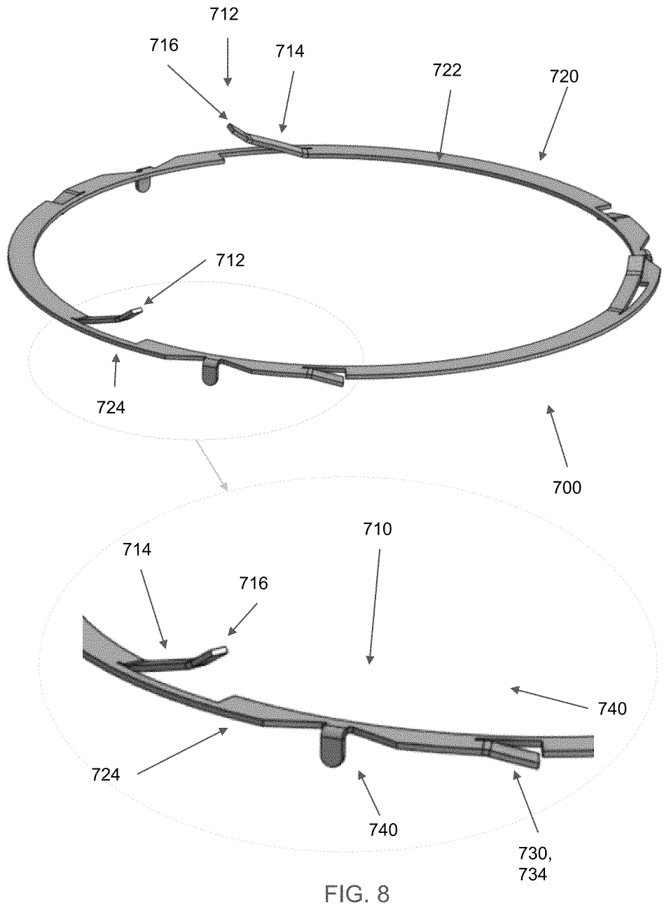

In order to ensure the electrical connection between the ECG module 692 and the second electrode 170, the ECG-watch 100 (and in particular the watchcase 110) comprises an electrical connector 700 shown in FIGS. 7 to 8 in particular, which ensures the electrical connection between the rotatable bezel 140 and the ECG module 692 (for example via a part of the watchcase 110). The electrical connector 700, being mounted on the watchcase 110, does not rotate, unlike the rotating bezel 140. Beneficially, the electrical connector 700 is removably mounted on the watchcase 110. In particular, as explained below, the electrical connector 700 is not welded or glued to the watchcase 110 but simply cooperates mechanically. The electrical connector 700 is therefore easily replaceable in case of wear.

In an embodiment, the electrical connector 700 is made of a conductive material, such that the entire electrical connector 700 may conduct current or transmit potential.

The electrical connector 700 may comprise at least one spring 710, for example a compression spring. In the embodiment illustrated in FIGS. 7 and 8 in particular, a plurality of compression springs 710 are provided, evenly distributed (e.g., three, distributed at 120° about the Z direction). In particular, the compression spring 710 is compressed by the bezel 140, so that with each rotation of the bezel 140, the compression spring 710 allows contact to be maintained at least when the bezel 140 is in a step (desirably at any time). The rotating spring 710 experiences friction, repeated impact, strain fatigue. When a plurality of compression springs 710 is provided, they may be mechanically synchronized (all in the same state for each bezel position) or they may be desynchronized (some more or less compressed). The benefit of a plurality of springs is to ensure mechanical (and electrical in our case) redundancy, which improves the reliability of the ECG watch.

Several embodiments of the compression spring 710 will be described.

As illustrated in FIGS. 7 and 8, the compression spring 710 may be a leaf spring 712. Compression is provided by the bezel 140 positioned above (in the Z direction) the electrical connector 700 and the bezel holder 630 positioned below (in the Z direction), such that contact between the bezel 140 and the electrical connector 700 is maintained at all times. The electrical connector 700 comprises a ring 720, configured to extend around (in XY projection) the glass 130 or dial 132. The ring may be in the form of a flat leaf spring. The thickness of the ring may be between 0.05 mm and 1 mm, or between 0.1 mm and 1 mm, and in an embodiment between 0.2 mm and 0.3 mm). The springs 710 may extend from a first side 722 of the ring 720. On a second side 724 of the ring 720, there may be one or more feet 730 for holding the electrical connector 700 stable and providing electrical conduction. In order to maximize the flatness of the electrical connector 700, the ring 720 is flat. Similarly, on the second side 724 may be one or more legs 740. The feet 730 and the legs 740 will be described in more detail below. The redundancy of the contacts allows a contact to be maintained even if one of the contacts should break.

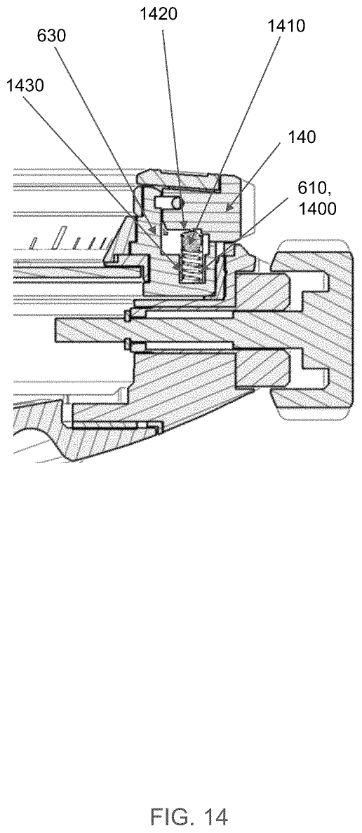

FIG. 9 shows an isolated view 900 of the bezel 140 and in particular of the main body 650 of the bezel 140. In particular, the outer side face 144 and the bottom face 902 are visible therein. The bottom face 902 faces the electrical connector 700 and mechanically cooperates with the latter. In particular, as illustrated, the bezel 140 comprises a series or succession of notches 910 on the bottom face 902, which participate in the step-by-step movement of the bezel (one notch for one step). The notches 910 may extend around the entire circumference of the lower face 900, so that there is no dead zone during the rotation of the bezel 140. The notches 910 are engaged by the spring 710 when it is in compression (or by a part pushed by the spring). The function of this engagement is twofold: the first is to block (at least limit) the rotation of the bezel 140, for example according to a pawl or ratchet system (notch and leaf spring), as illustrated in FIGS. 9, 10 and 11, or according to a cam/follower system (lobed cam and ball bearing for example, as illustrated in FIG. 14).

Depending on the shape of the notches 910, the rotation may be clockwise or counterclockwise only. The orientation of the leaf spring 712 (which extends from the ring in a clockwise or counterclockwise direction—relative to the Z direction) may define the direction of rotation of the bezel 140. A leaf spring 712 extending counterclockwise may block clockwise rotation or vice versa (provided the detent 910 is designed to block rotation, as described in the next paragraph).

On the inner side face 904 may be a groove or channel 906, configured to receive a retaining ring 1500 which will be described below.

In FIG. 9, each notch 910 is formed by two faces 912, 914. The two faces 912, 914 may have equal slopes, so that the notch is symmetrical (not shown). Two successive notches are separated by an edge 916. If the faces 912, 914 have a sufficiently steep slope, it is the orientation of the leaf spring 712 that defines the direction of rotation of the bezel 140. Alternatively, as shown in the figures, the slope of face 914 may be steeper (e.g., vertical or near vertical) than that of face 912, so that leaf spring 712 abuts face 914 of a notch 910 to block rotation in one direction.

The edge 916, which separates the two faces 912, 914 may have a flat or rounded shape to limit wear of the leaf spring 712 with each rotation of the bezel 140.

With less pronounced notches 910 (lower slopes 912, 914) or a flatter leaf spring 712, it is possible to have a bezel 140 mounted for clockwise and counter-clockwise rotation, with the notches simply defining stable positions.

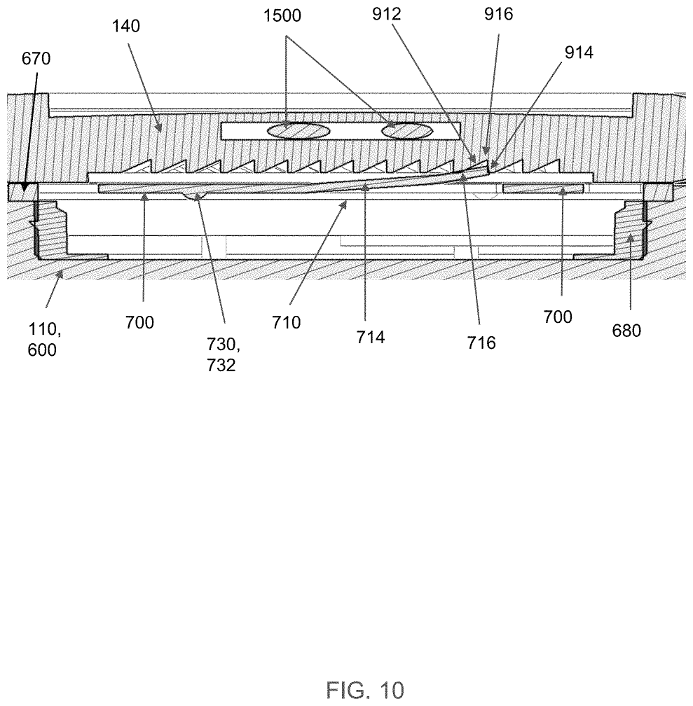

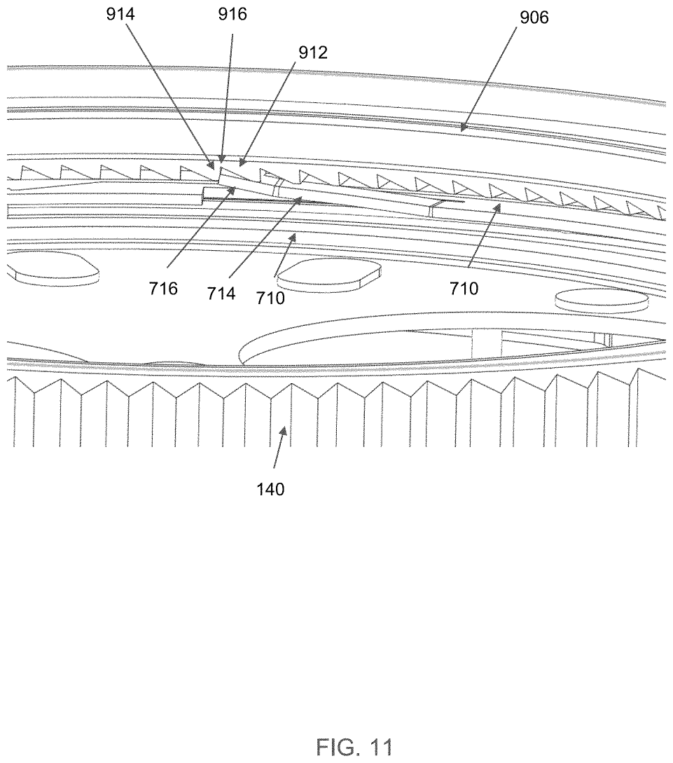

As seen in FIGS. 7 and 8, 10 and 11, the leaf spring 712 may include a first portion 714, which extends from the ring 720 at a first slope and then a second portion 716, at a second slope steeper than the first slope (at rest or relative to the plane of the ring 720—which generally coincides with an XY plane), which extends from the end of the first portion 714. This double inclination optimizes the engagement between the notches 910 of the bezel 140 and the electrical connector 700. Indeed, as seen in FIGS. 10 and 11 representing the engagement of the leaf spring 712 in a notch 910 of the bezel 140, the leaf spring 712 has a length much greater than the distance between two successive notches 910 (length of the leaf spring along the periphery greater than at least three notches or even five, and for example between four notches and seven notches). The second slope of the second portion 716 also facilitates the locking of this second portion 716 of the leaf spring 712 in the notch 910. The second portion 716 also plays a role in the haptic sensation of rotation of the bezel 140. The benefit of having a leaf spring 712 extending over a plurality of notches is to limit the angular displacement (the angle of the first slope in particular) and the deformation of the leaf spring 712 each time the bezel 140 is rotated past a notch, and thus to keep the material in an elastic deformation range (regardless of the presence or absence of the two portions 714, 716). In addition, the length of the leaf spring also determines the overall tilt and allows for better rotation lock by aligning the rotational force with the direction of the first portion 714 of the leaf spring 712. These optimizations limit wear and tear on the electrical connector 700, whose role as an electrical connection between the second electrode 170 and the ECG module 692 is critical to the proper functioning of the ECG measurement even after several thousand rotations of the bezel 140.

In an embodiment, illustrated in FIGS. 10 and 11 in particular, the second portion 716 of the leaf spring 712 may rest at least partially on the slope 912 of the notch (point contact, line contact or even surface contact if the slopes are similar), which is the lower slope, and abuts against the slope 914 of the same notch, which is the higher slope. This configuration allows for a first portion 714 with a low slope and thus to be slightly displaced, as explained above.

In an embodiment not shown in the figures, the leaf spring has a single straight or curved portion. In order to maintain a leaf spring length greater than several (e.g., three or five) notches while maintaining a leaf spring engagement with a notch, the depth of the notches in the bezel may be decreased from the configuration with the portion 716 more angled than the portion 714.

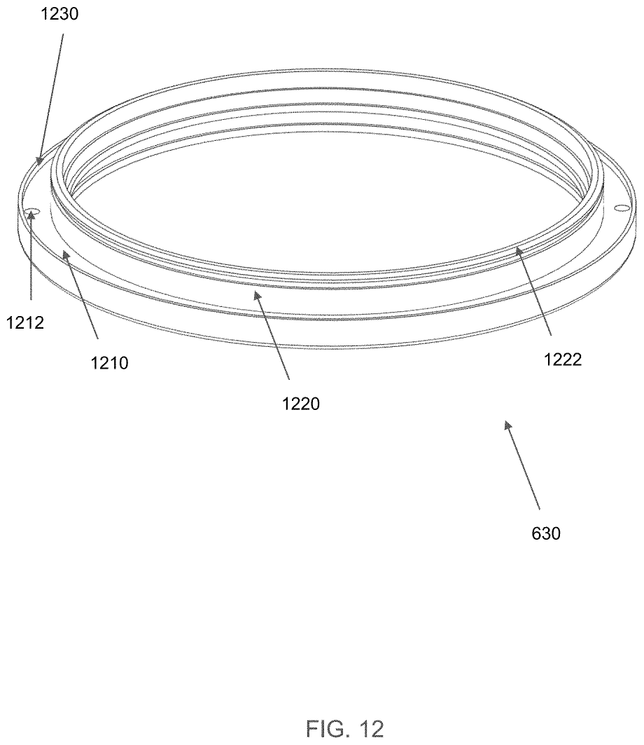

The watchcase 110 may further comprise a bezel holder 630, introduced in connection with FIG. 6 and further illustrated in FIG. 12. In particular, the bezel holder 630 receives and carries the rotatable bezel 140. Forming part of the watchcase 110, the bezel holder 630 is fixed and is configured to guide the bezel 140 in rotation. In the illustrated embodiment, the bezel holder 630 comprises a ring 1210, an inner cylindrical wall 1220, extending along the Z-direction (toward positive Z typically) from an inner edge of the ring 1210, and an outer cylindrical wall 1230, extending from along the Z-direction (toward positive Z typically) from an outer edge of the ring 1210. Radially inwardly of this inner cylindrical wall 1220 is the dial 132 (in the case of a hybrid ECG-watch). The inner cylindrical wall 1220 may include a groove 1222, on an outer side of the inner cylindrical wall 1220 (i.e., the side of the inner cylindrical wall 1220 that faces the outer cylindrical wall 1230), for receiving a retaining ring 1500 (shown in FIGS. 6, 13, and 15 in particular). The groove 1222 of the bezel holder 630 is opposite the groove 906 of the bezel 140 (visible in FIGS. 6, 9, 11 and 12), thus defining an annular volume Va within which the holding ring 1500 is located. This part will be described below. The bezel holder 630, more specifically the ring 1210, may also include one or more locking holes 1212.

As previously indicated, the bezel holder 630 rests on the retaining joint 680, visible in FIGS. 6 and 10, which interfaces the main body 600 of the watchcase 110 and the bezel holder 630.

The electrical connector 700 is positioned on the ring 1210, between the inner cylindrical wall 1220 and the outer cylindrical wall 1230. The ring 720 of the electrical connector 700 is arranged parallel to the ring 1220 of the bezel holder, spaced apart by the presence of the feet 730.

The electrical connector 700 establishes an electrical connection between the bezel 140 and the ECG module 692. In particular, the electrical connector 700 is electrically connected with the bezel holder 630 via, inter alia, the feet 730, which act as localized electrical contactors. By localized, it is meant that the electrical contact is made at a location specifically provided by the designers of the ECG-watch. By concentrating the contact in a small area, the contact force between the electrical connector 700 and the bezel holder 630 is increased. This reduces the contact resistance. Since the electrical chain plays a critical role in the quality of the ECG signal, simple metal-to-metal contacts between two planar portions (e.g., ring 720 of the electrical connector 630 and ring 1210 of the bezel holder 680) may generate noise. By using localized electrical contacts (point or line contact, but over a distance of a few millimeters maximum and precisely established), the electrical chain is more stable.

The presence of a plurality of feet 730 on the second side 724 of the ring 720 of the electrical connector 700 ensures electrical continuity even if micro-displacements should occur. The feet 730 may be evenly distributed along the periphery of the electrical connector 630. Between two and ten feet (six in FIGS. 7 and 8) may be provided.

The feet 730 further provide mechanical stability of the electrical connector 700 on the bezel holder 630. To this end, at least three (typically spaced apart, e.g., evenly spaced) feet 730 are provided. For example, between 3 and 10 feet may be provided, or between 4 and 7 feet, or 6 feet (distributed every 60 degrees). The bezel 140, when rotated by the user, generates a force on the spring or springs 710, which transmit the force to the feet 730. The mechanical stability of the electrical connector 700 as well as the force generated by the compression of the springs 710 contributes to the electrical stability of the ECG-watch. Typically, as many feet 730 as springs 710 are provided, in order to achieve maximum symmetry of the electrical connector 700.

FIGS. 7, 10, and 13 illustrate a first embodiment of the feet 730, wherein the feet 730 are lugs 732. The lugs 732 may have a partially spherical or hemispherical shape or more generally a rounded shape. Alternatively, the lugs may have a slightly flat end. In an embodiment, the lug 732 is solid (i.e., it does not deform under the forces involved with the bezel). The lug 732 may be made of the same material as the rest of the electrical connector 700, optionally with the same coating.

FIG. 8 illustrates a second embodiment of the feet 730, wherein the feet 730 are tabs 734 (or leaf springs). The tabs 734 may be leaf spring-like in shape extending at an angle (strictly less than 90°, for example at 10° and 45°) and thus from the ring 720 on the second side 724. In an embodiment, the tabs 734 are rigid, at least more rigid or significantly more rigid than the compression spring 710, such that rotation of the bezel 140 compresses the spring 710 and not the tab 734. In an embodiment, the tab 734 is slightly deformable to provide electrical contact. In an embodiment, the tab 734 is compressed upon assembly to seat the electrical connector 700 on the bezel holder 630.

In turn, the bezel holder 630 is electrically connected to the ECG module 692 via conductive components, for example with a conductive coating such as gold (gold-plated stainless steel). The bezel holder 630 may be made of a connector material, such as stainless steel (316L, 301, 304 or 446), with or without a special coating (such as gold).

As seen in FIGS. 6, 7, 8, the electrical connector 700 also comprises legs 740, already shown in connection with FIGS. 6-8. The legs 740 extend from the second side 724 (opposite side of the springs 710) of the ring 720. The bezel holder 630 comprises the locking hole 632, which is configured to receive the leg 740. In this way, the electrical connector 630 is locked against rotation about the Z-axis and may take up the torque that the bezel 140 exerts on the electrical connector 600 when it rotates or when it abuts the spring 710. In order not to adversely affect mechanical stability and electrical connection via the feet 730, the leg 740 is less long than the depth of the locking hole 632. In an embodiment, at least three legs 740 are provided, in order to symmetrize the electrical connector 700 and to be able to compensate for deformation or damage of one or two of them. More particularly, as many legs 740 as feet 730 as compression springs 710 are provided.

Other embodiments of the electrical connector will be described. In an embodiment illustrated in FIG. 14, the electrical connector 630 comprises a compression spring 710 (e.g. a coil spring 1400) with a ball bearing 1410. The bezel 140 then comprises on its inner side a cam 1420, for example lobed, on which the ball bearing 1410 rolls. The electrical connection is made via the ball bearing 1410 and possibly the spring 710, 1400. The spring 710, 1400 may be housed in a hole 1430 in the watchcase 110, for example in the bezel holder 630 (the ring 1210 of the bezel holder 630, as shown) or the main body of the watchcase 600 directly, to hold the spring in position. The shape of the cam 1420 allows for unidirectional or bidirectional (clockwise and/or counterclockwise) stepwise rotation. The shape of the cam 1420 also determines the force to be applied to drive the bezel 140 in rotation. As the bezel 140 moves, the ball bearing 1410 rolls on the cam 1420 of the bezel 140 and compresses the spring 610 along the Z direction. Alternatively, the spring 1400 may be inclined. In particular, this arrangement allows for unidirectional rotation. The ball bearing 1410 may be coated with a conductive coating such as gold. The spring may also be coated with a conductive coating such as gold. Alternatively, the spring 1400 serves only to manage the rotation of the bezel and leaf springs 700, as described above, provide the electrical function. Alternatively and conversely, the spring 1400 serves only to manage the electrical connection and leaf springs 700, as previously described, manage the rotation of the bezel.

In an embodiment not shown, the electrical connector 700 may include a brush configured to slide over the rotating bezel. In order to provide a detent rotation, a pawl system may be provided, for example, with leaf springs similar to the leaf springs 710 or a helical spring 1400 with a ball bearing 1410. The brush may be electrically connected to the bezel holder. The brush may also include a compression spring to ensure contact between the brush and the bezel.

The electrical connector 700 is formed from a conductive material. Alternatively or additionally, the electrical connector 700 is coated with a conductive material (conductive coating). The coating may have a better conductivity than the material of the electrical connector 700. In an embodiment, the electrical connector is made of a conductive material (e.g., stainless steel, phosphor-bronze, etc.) and the leaf springs 710 or the entire electrical connector 700 are coated with another conductive material (e.g., gold, nickel, etc.). In an embodiment, the electrical connector 700 is made of metal such as steel, for example stainless steel, such as steel 301 (fatigue resistant steel), or phosphor-bronze and is at least partially and desirably entirely covered with a coating, for example a metallic coating such as gold or nickel. Alternatively, the connector 700 is plastic with a conductive coating as mentioned above. The bezel holder 630 may be made of a material identical to the electrical connector 700.

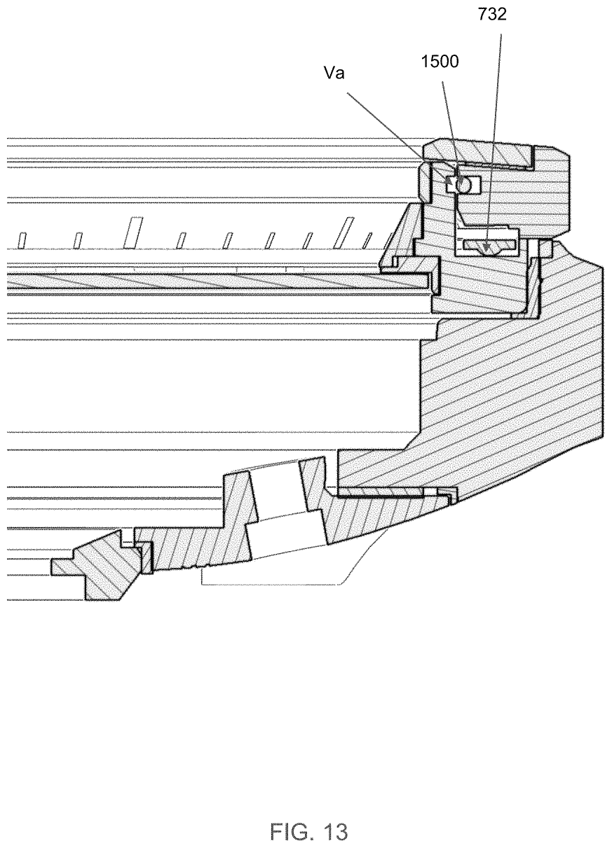

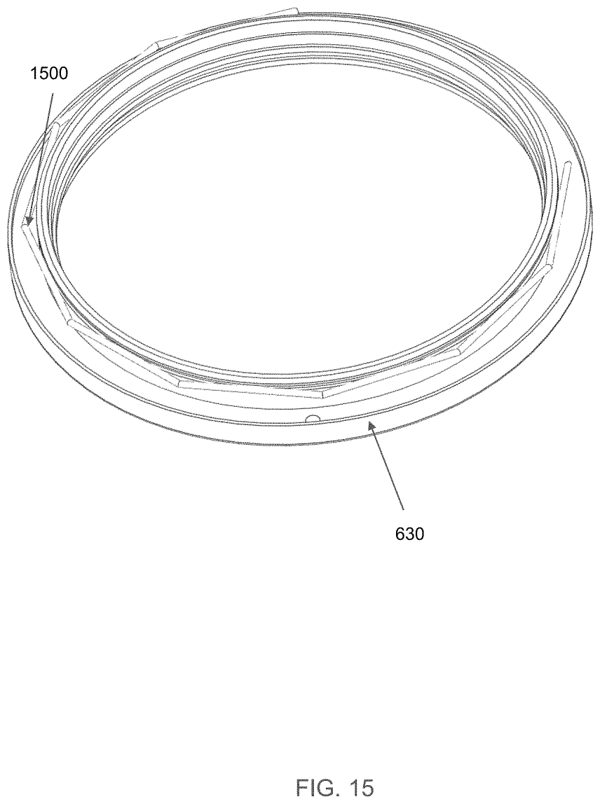

As previously mentioned, the ECG-watch comprises a retaining ring 1500, visible in FIGS. 6, 10, 13, 14, 15, which serves to block Z-directional movement of the bezel 140 relative to the watchcase 110. The retaining ring 1500, shown in FIG. 15 (which also depicts the bezel holder 630), may be a rigid polygonal rod extending at least 270 degrees into the annular volume. Because of its polygonal rather than circular shape, the retaining ring 1500 is accommodated, depending on the angular position along the annular volume Va, alternately in the groove 906 of the bezel 140 and in the groove 1222 of the bezel holder 630, thereby blocking the movement of the bezel 140 in the Z-direction. In FIG. 13, the retaining ring 1500 is between the two; in FIG. 6, the retaining ring 1500 is at the bottom of the groove 1222 of the rotatable bezel 140. Because of its polygonal shape, the retaining ring 1500 does not occupy the entire annular volume Va, which limits friction with the bezel 140 during rotation. The retaining ring 1500 may be slightly compressed during assembly of the bezel 140 and may remain slightly compressed once in position within the annular volume Va.

Alternatively, a retaining ring extending into both grooves at the same time may be provided, but the frictional surface is increased compared to the polygonal hoop, which may impede the rotation of the bezel.

The serrations 148 on the outer side face 144 of the bezel 140 (or bezel body 650), which thus form part of the second electrode 170, allow for improved electrical contact with the users skin (the serrations penetrate the skin, thereby decreasing the contact resistance and providing a peaking effect at the serrations. The performance of the ECG watch may thus be improved.

The ECG-watch 100 is typically a hybrid watch with hands. The hands are driven in rotation by one or more micromotors, controlled by the control unit 696.

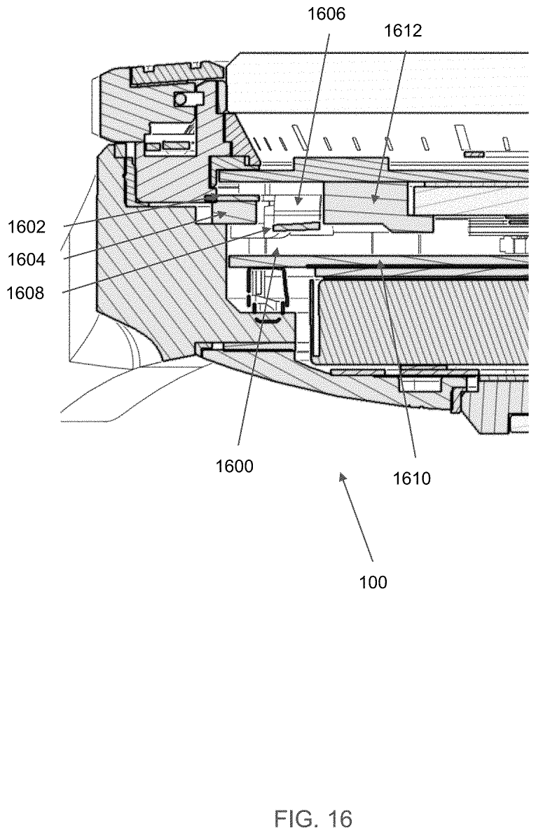

As previously described, the electrical chain through which the ECG electrical signal passes comprises the electrode 170 of the bezel 140, then the electrical connector 700, then the bezel holder 630, then an intermediate electrical connector 1600 (see FIG. 16), then a spring 1700 (see FIG. 17), then the electronic board 1610 (see FIG. 16) on which the ECG module 692 and/or the control unit 696 is mounted. The intermediate electrical connector 1600 will be described below, in FIGS. 16 and 17. To prevent short circuits, the contact chain is electrically isolated from parts that do not perform an electrical function for the ECG sensor (such as the main body 600 of the watchcase 110, for example). A holder member 1612 is shown in FIGS. 16 and 17, which is used to hold certain components of the ECG-watch 100 in place, including the intermediate electrical connector 1600. This holder member 1612 is typically made of plastic and may have a disk shape with indentations for receiving components.

The electrical chain between the bezel holder 630 and the ECG module 692 may therefore include the intermediate electrical connector 1600, shown in FIGS. 16 and 17, which connects the bezel holder 630 to the electronic board 693. The intermediate connector 1600 may include a plate 1602 (a flat piece, with a banana-shaped contour), which contacts the bezel holder 630. To ensure contact, compression is instituted during installation, with a foam 1604 positioned under the tray 1602, which is compressed. The foam 1604 also provides electrical insulation. From the tray 1602 extends an angled three-dimensional arm 1606 (the sloped face of which is visible in FIG. 16) that extends toward the case back 120. At the end of the arm 1606 is another plate 1608, parallel to the plate 1602. The plate 1608 faces the electronic board, referenced 1610. FIG. 17 illustrates a 90° view of FIG. 16. The angled three-dimensional arm 1606 is visible, as is the slope (but seen from the side in cross-section this time). The plate 1602 is out of the cross-sectional view. A spring 1700, in compression, is positioned between the plate 1608 and the electronic board 1610 to ensure electrical contact. Finally, various components, such as the control unit 696 and/or the ECG module 692, are also shown on the electronic board 1610 and are electrically connected to the spring 1700 by conductive circuit boards.

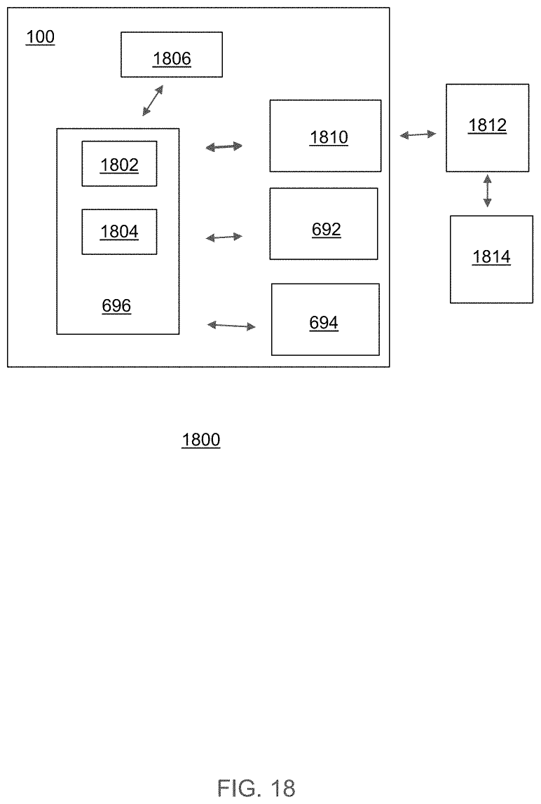

As shown schematically in FIG. 18 which illustrates a diagram 1800, the control unit 696 may include one or more processors 1802 and a memory 1804 that stores instructions suitable for execution by the processor 1802. The memory 1804 is a non-transitory memory. The one or more processors are implemented using electronic circuitry.

The ECG-watch 100 may also include an accelerometer 1806, connected to the control unit 696 (for tracking sleep, activity, etc.).

To supply the various components with electrical power, the ECG-watch 100 comprises a battery 1808, such as a battery or a rechargeable battery. The previously described recess 640 allows the case back 120 to be placed on a charging station to recharge the battery 1808.

The ECG-watch 100 comprises a wireless communication module 1810, such as a Bluetooth® or Bluetooth® Low Energy module or a Wi-Fi module (Wi-Fi being a wireless network protocol, based on the IEEE 802.11 family of standards) or a cellular module (GSM (Global System for Mobile communication), 2G (2^{nd} generation cellular network), 3G (3^{rd} generation cellular network), 4G (4^{th} generation cellular network), 5G (5^{th} generation cellular network), Sigfox (which is a wireless network to connect low-power object such as sensors and devices), etc.), which allows it to communicate bidirectionally with at least one external terminal 1612, such as a mobile phone. The external terminal 1812 may then communicate (bidirectionally) with a remote server 1814 for data storage and processing. Alternatively or additionally, the wireless communication module 1810 may communicate directly with the remote server 1814, such as via the cellular network or via a Wi-Fi network. Data obtained by the ECG-watch 100, such as an electrocardiogram, but also indications of heart rate, activity or oxygen saturation, are transmitted to the external terminal 1812 via the wireless communication module 1810. The control unit 696 may process certain signals before sending them, to limit the size of the data.

The PPG module 694 and the ECG module 692 are also connected to the control unit 696 or are integrated and/or partially integrated therein. The ECG module 692 is known per se and will not be described in detail. Various types of electronic components may be included in the ECG module 692 (processor, resistor, capacitor, etc.) to carry the functions of the ECG.

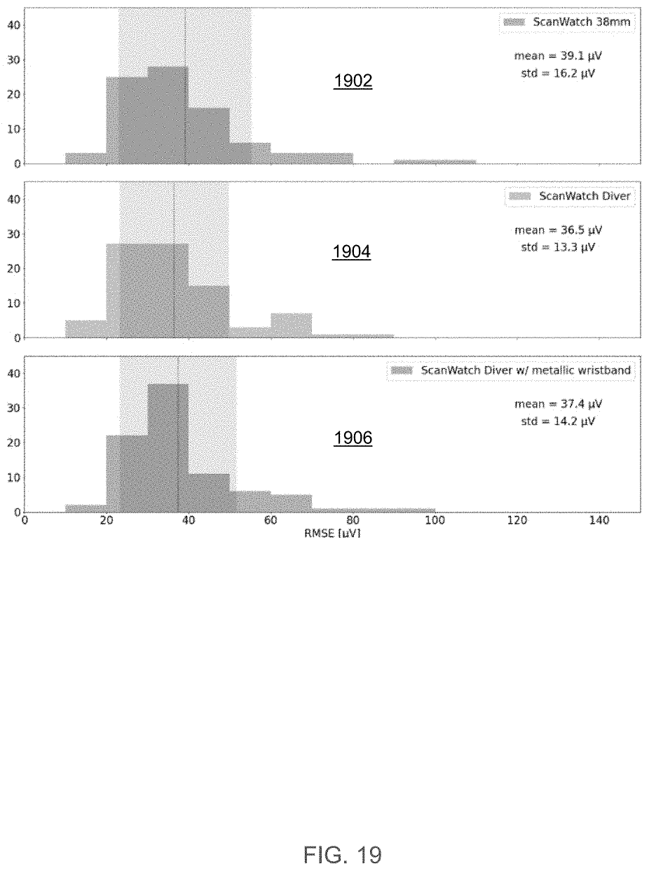

FIG. 19 shows the performance results of the ECG-watch 100. In FIG. 19, the y-axis of the graphs represents the number of ECG recordings (86 in total) made with a watch and the x-axis represents the mean squared error (MSE) of a ScanWatch 38 mm (graph 1902), of an ECG-watch 100 according to the embodiment of FIGS. 1-3, 5-7, 9-15 with a non-conductive strap (graph 1904) and a metallic strap (graph 1906), with respect to a reference electrocardiogram (Schiller Cardiovit FT-1). “Mean” means the average and “std” means the standard deviation. It may be observed that the results obtained by the ECG-watch 100 are no worse than those obtained by the ScanWatch 38 mm, which in 2019 obtained CE certification. Regarding the wear tests (two thousand rotations of the bezel), the following results were obtained:

For the SNR (signal-to-noise ratio):
unworn ECG-watches 100: 19.0 (±3.5) dB for the ECG-watch 100 with the steel bezel and 19.2 (±3.4) dB for the ECG-watch 100 with the titanium bezel;
worn ECG-watches 100: 17.8 (±3.6) dB for the ECG-watch 100 with the steel bezel and 18.0 (±3.5) dB for the ECG-watch 100 with the titanium bezel.

For the signal:
unworn ECG-watches 100: 725.9 (±261.6) dB for the ECG watch 100 with the steel bezel and 705.7 (±258.7) dB for the ECG-watch 100 with the titanium bezel;
worn ECG-watches 100: 702.9 (±259.4) dB for the ECG watch 100 with the steel bezel and 732.5 (±283.6) dB for the ECG-watch 100 with the titanium bezel.

For noise:
unworn ECG-watches 100: 29.1 (±14.7) dB for the ECG watch 100 with the steel bezel and 27.4 (±11.0) dB for the ECG-watch 100 with the titanium bezel;
worn ECG-watches 100: 32.9 (±10.9) dB for the ECG watch 100 with the steel bezel and 31.7 (±10.1) dB for the ECG-watch 100 with the titanium bezel.

In addition, it was observed that no additional noise that could interfere with the ECG analysis occurred. The test results showed that the ECG-watch 100 as presented in the description gave good ECG results and that its resistance to wear was good.

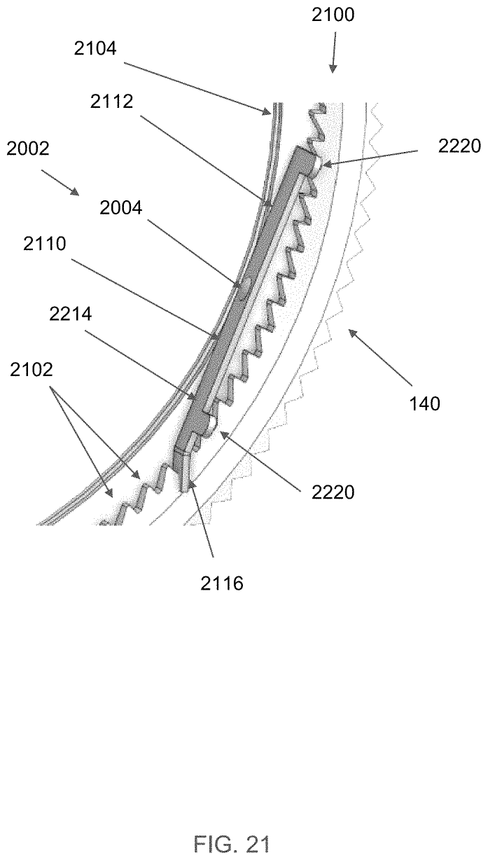

Another embodiment of the electrical connector and the rotative bezel will be described below with reference to FIGS. 20 and 21.

As seen in FIG. 20, the electrical connector comprises a compression spring which is a leaf spring 2002 in contact with the inner cylindrical wall 1220 of the bezel holder 630. Beneficially, the electrical connector comprises a plurality of compression springs 710 each in the form of a leaf spring 2002 in compression. The leaf springs 2012 are angularly spaced, beneficially regularly. Here, the electrical connector 700 comprises three leaf springs 2012 spaced approximately 120° apart (i.e. +1-3°). In the illustrated embodiment, the electrical connector 700 comprises a plurality of independent parts. Alternatively, the electrical connector 700 may include a ring that connects the plurality of blades.

Each leaf spring 2002 comprises a foot 2004, more visible in FIG. 20, for holding the electrical connector stable and for ensuring electrical conduction with the bezel holder 630. In particular, each foot 2004 is adapted to cooperate with the inner cylindrical wall 1220 of the bezel holder 630.

As illustrated in FIG. 21, the bezel 140 comprises a series or succession of notches 910 arranged on the inner side face 904, which participate in the step-by-step movement of the bezel (one notch for one step). The notches 910 may extend all around the inner lateral face 904, so that there is no dead zone during the rotation of the bezel 140.

The leaf springs 2002 are adapted to engage in the notches 2102. To this end, each leaf spring 2002 comprises a first part 2110 and a second part 2112 extending on either side of the foot 2004, both portions extending substantially tangentially to the inner cylindrical wall 1220. For example, the two parts extend along a similar length on either side of the foot 730.

The first part 2110 may comprise a first portion 2114 (similar to first portion 714), which extends from the foot 730 at a first slope and then a second portion 2116 (similar to second portion 716), at a second slope steeper than the first slope (at rest or relative to the inner cylindrical wall 1220), which extends from the end of the first portion 2114 and is adapted to engage a notch 2102, as seen in FIG. 21.

Each leaf spring 2002 may include at least one leg 2220 extending from an edge of the leaf spring 2002, orthogonal to the XY plane. Here, two legs 2220 are provided, one leaf spring extending from each portion 2110, 2112. Each leg 740 is suitable for insertion into a locking hole 632 arranged in the bezel holder 630 (see FIG. 20). The electrical connector 700 is thus locked against rotation about the Z-axis and may take up the torque that the bezel 140 exerts on the electrical connector 700 when it rotates or when it abuts against the spring 710.

In the embodiment of FIGS. 20 and 21, the compression spring works in a direction transverse to the Z axis, and more particularly in a direction of the XY plane, which set the assembly free from tolerances along the Z axis. Assembly tolerances along the Z axis may lead to assembly variances and could, in fine, affect the quality of the ECG signals. In the embodiment of FIGS. 7 and 8, the compression spring works parallel to the Z axis.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

It will be appreciated that the various embodiments described previously are combinable according to any technically permissible combinations.

1. A portable electronic device configured to be positioned on a users wrist, the portable device being configured to perform an electrocardiogram, ECG, said portable electronic device comprising:
a watchcase,
a case back, configured to be at least partially in contact with the skin of the wrist,
a glass,
a bezel, mounted on the watchcase and surrounding the glass, movable in rotation with respect to the watchcase,
a first ECG electrode, made of conductive material, on the case back and configured to be in contact with the skin of the wrist,
a second ECG electrode, made of conductive material, on the bezel,
an ECG electronic module, electrically connected to the first ECG electrode and the second ECG electrode, and configured to receive and process electrical signals from a user and retrieved by the ECG electrodes, to perform an electrocardiogram.

2. The portable electronic device according to claim 1, wherein the bezel comprises a bezel body and the second ECG electrode is formed by the bezel body, such that the entire bezel body forms the second electrode, and any portion of the bezel body is touchable to take an ECG measurement.

3. The portable electronic device according to claim 1, wherein the second ECG electrode is electrically connected to the ECG electronic module regardless of an angular position of the bezel.

4. The portable electronic device according to claim 1, further comprising an electrical connector in the watchcase configured to electrically connect the bezel and the ECG electronic module, the electrical connector providing an electrical connection between the ECG electronic module and the second ECG electrode.

5. The portable electronic device according to claim 4, wherein the electrical connector is removably mounted to the watchcase.

6. The portable electronic device according to claim 4, wherein the electrical connector comprises at least one compression spring configured to make electrical contact with the bezel.

7. The portable electronic device according to claim 6, wherein the at least one compression spring is a leaf spring.

8. The portable electronic device according to claim 6, wherein the electrical connector comprises a plurality of compression springs, the compression springs being angularly spaced.

9. The portable electronic device according to claim 6, wherein the electrical connector comprises feet, wherein the feet are electrically connected with the ECG electronic module, such that an ECG signal passes through at least one of said feet.

10. The portable electronic device according to claim 9, wherein each compression spring exerts pressure on the feet so as to maintain the electrical connection of the feet to the ECG electronic module.

11. The portable electronic device according to claim 9, wherein the electrical connector comprises between 3 and 10 feet.

12. The portable electronic device according to claim 9, wherein the electrical connector comprises as many feet as compression springs.

13. The portable electronic device according to claim 9, wherein the electrical connector comprises a ring positioned around the lens.

14. The portable electronic device according to claim 13, wherein the at least one compression spring extends from one side of the ring and the feet extend from another side of the ring.

15. The portable electronic device according to claim 9, wherein the feet are tabs, the tabs being stiffer than the at least one compression spring.

16. The portable electronic device according to claim 6, wherein the at least one compression spring works parallel to an axis of rotation of the bezel.

17. The portable electronic device according to claim 6, wherein the at least one compression spring works orthogonal to an axis of rotation of the bezel.

18. The portable electronic device according to claim 6, wherein the bezel comprises, on an inner face, a series of notches adapted to interact with the at least one spring in compression, the series of notches and the spring enabling a stepwise displacement to be defined for rotation of the bezel.

19. The portable electronic device according to claim 4, wherein the electrical connector comprises a conductive coating.

20. A method of taking an electrocardiogram, ECG, using a device according to claim 1, the method comprising, receiving a first signal from the first ECG electrode in contact with an arm of the user and receiving a second signal from the second ECG electrode in contact with another arm of the user.
